(19) United States
(12) Patent Application Publication
(10) Pub. No.: US 2003/0165487 A1
(43) Pub. Date: Sep. 4, 2003

(54) Amylase feed supplements for improved ruminant nutrition

(76) Inventors: Juan Tricarico, Lexington, KY (US); Karl A. Dawson, Lexington, KY (US); Jay D. Johnston, Kanata (CA)

Correspondence Address: GREENLEE WINNER AND SULLIVAN P C, 5370 MANHATTAN CIRCLE, SUITE 201, BOULDER, CO, 80303, US

(21) Appl. No.: 10/366,602

(22) Filed: Feb. 12, 2003

Related U.S. Application Data
(60) Provisional application No. 60/356,324, filed on Feb. 12, 2002.

(51) Int. Cl. A61K 38/47

(52) U.S. Cl. 424/94.61

(57) ABSTRACT

The present disclosure provides methods and compositions for the following: improving rumen fermentation efficiency; increasing the efficiency with which dietary starch is utilized while preventing a deleterious increase in ruminal lactic acid concentration and/or a deleterious drop in ruminal pH; and promoting the growth of beneficial ruminal microorganisms. As a result, there is a concurrent improvement in the productivity and profitability of ruminant animal husbandry, especially in the diary and beef industries. These methods and compositions provide for the supplementation of the diet of a ruminant animal with exogenous amylase at relatively low levels, without any concomitant need for exogenous fibrolytic enzymes such as cellulases and xylanases. The methods and compositions of the present invention can further comprise supplementation with yeast preparations, rumen buffering agents, ionophores or other growth and productivity stimulating agents.

### CROSS REFERENCE TO RELATED APPLICATIONS

This application claims benefit of U.S. Provisional Application No. 60/356,324, filed Feb. 12, 2002, which application is incorporated herein by reference.

### ACKNOWLEDGMENT OF FEDERAL RESEARCH SUPPORT

not applicable.

### BACKGROUND OF THE INVENTION

The field of the present invention is animal husbandry, especially as related to ruminant nutrition. Specifically, the present invention relates to supplementation of the diet of domesticated ruminant animals with amylase(s) at a level sufficient to improve the performance of the animals without resulting in deleterious effects due to a too great increase in the rate and extent of starch metabolism in the rumen. In particular, the supplementation of the feed rations of lactating dairy cattle results in increased milk production and/or fat content without an increase in the total feed rations; similarly, supplementation of feed rations of beef cattle with amylase at the levels taught herein results in improved weight gain.

Ruminant animals of particular economic importance include cattle, sheep, buffaloes and goats. Others include camels, guanaco, llamas, wapiti, antelope, musk oxen, giraffes and others.

The digestive tract of ruminants includes the reticulum, rumen, omasum, abomasum, small intestine, cecum, colon and rectum. Rumination results in increases in the surface area of feed particles and increased salivation, which contributes to maintenance of advantageous rumen pH. Muscular contractions within the rumen mix newly ingested feed particles with the rumen contents and wash the epithelium of the digestive system with volatile fatty acids (VFA) produced by the rumen flora; these VFA are absorbed through the rumen wall and serve as the primary energy source for the ruminant animal.

The rumen is an anaerobic environment where substrates are incompletely oxidized. NADH production and reoxidation is a critical feature of the fermentation in the rumen. Acetate is the most abundant end product of ruminal fermentation. Carbohydrates are also metabolized to propionate, butyrate and lactate.

The flora of the rumen include large numbers of bacteria, and these bacteria contribute to the degradation of high molecular weight materials as well as transformations of simple molecules. Cellulolytic rumen bacteria include Ruminococcus albus, Ruminococcus flavifaciens, Butyrivibrio fibrisolvens and Fibrobacter succinogenes. Megasphaera elsdenii, Peptostreptococcus anaerobius and Selenomonas ruminantium metabolize the products of other bacteria to VFA.

Protozoa are an important part of the overall rumen community; they can constitute up to half of the microbial mass. Although not essential to the animal's nutritional state, protozoa participate in the digestion of fiber, and they can sequester starch granules, thereby modulating the fermentation rate.

Fungi are another key component of the rumen flora and fauna, and the anaerobic fungi secrete extracellular enzymes which break down cellulose, xylans, polygalacturonic acid polymers and the like. Important rumen fungi include Neocallimastix, Orpinomyces and Piromyces species.

In nature, ruminants live on forage materials, with relatively low grain intake. However, high levels of animal productivity are not maintained by forage. In developed countries the fiber-rich forage diet of ruminants is commonly supplemented with grain. Various strategies have been employed to improve utilization of dietary materials and improve the economics of the production of milk and meat.

Enzymatic supplements have been added to the diets of ruminant farm animals. With respect to improving or increasing the digestion and metabolism of starch, there is a need for caution so that the rumen ecology is not disturbed such that the animal suffers deleterious effects. For example, a sudden and strong increase in starch degradation can result in a bloom of lactic acid producing bacteria (Streptococcus bovis and Lactobacillus spp.) with a concomitant substantial drop in rumen pH, resulting in inflammation, potential infection and release of proteases into the animal's circulation. Certain starch-fermenting bacteria can produce polysaccharides which interfere with release of gaseous products from the rumen through eructation.

Starch digestibility is a factor which contributes to performance and profitability, especially in high producer dairy cows. Variation in the starch content of grains and starch digestibility is reflected in animal performance. Improved starch utilization is necessary to maintain high levels of milk production. Increased ruminal starch digestibility leads to increased total starch digestibility, and it has also been reported to increase microbial protein synthesis and microbial protein flow to the small intestine [Herrera-Saldana et al. (1990) J. Dairy Sci. 73:142]. Lykos et al. [Lykos et al. (1997) J. Dairy Sci. 80:3341] showed improved performance in dairy cows fed total mixed rations (TMR) with high rates of ruminal starch degradation. The relatively high levels of starch and free glucose in the feces of cattle fed coarsely and finely ground corn [San Emeterio et al. (2000) J. Dairy Sci. 83:2839] suggest that starch utilization in dairy cows and other ruminant farm animals can be improved.

There is a longfelt need in the art to improve agricultural productivity and profitability, especially with respect to domesticated ruminant animals including, but not limited to, lactating dairy cows and beef cattle. The present invention improves nutrient utilization in such animals without compromising animal health.

### SUMMARY OF THE INVENTION

The present invention provides a method for improving performance of domesticated ruminant animals, especially bovines, and as particularly advantageous, improving milk production in lactating dairy cows by improving starch utilization. The method comprises the step of adding at least one amylase to the diet of the ruminant animals. The amylase confers a beneficial effect on the nutritional status and improves performance and profitability of the ruminant animal even in the absence of additional exogenously supplemented enzyme activities such as xylanase and/or cellulase. As specifically exemplified herein, amylase produced by Aspergillus oryzae is fed to the dairy cows at a rate of from about 2000 to about 20,000 FAU units of amylase activity per cow per day, desirably from about 4000 to about 18,000 units per cow per day, or most desirably from about 5000 to about 10,000 units per cow per day. The amylase can be added to the feed to yield a specific activity of 600 units per gram of enzyme product. Assuming dairy cow intake of 20 kg of dry matter (DM) per day, this amounts to 0.3 units of enzyme per kg of DM consumed.

Another aspect of the present invention is a method for improving rumen fermentation efficiency, especially with respect to a fibrous diet with grain and starch-containing supplementation of the diet. This method comprises the step of administering exogenous carbohydrates, protease or amylase to the ruminant animals. As specifically exemplified herein, amylase produced by Aspergillus oryzae is fed to lactating dairy cows or pregnant cattle (especially dry dairy cows) or grain-fed beef cattle at a rate of from about 2000 to about 20,000 units of amylase activity per cow per day, desirably from about 4000 to about 18,000 units per cow per day, or from about 5000 to about 10,000 units per cow per day.

It is a further aspect of the present invention to improve performance in domesticated ruminant animals as set forth above by modulating the rumen ecology such that the growth of the desirable bacteria is stimulated and excessive growth of the less desirable bacteria, especially those which produce lactic acid, does not occur. This is accomplished by supplementing the diet of a domesticated ruminant animal, which eats grain or another starch source, with alpha amylase in an amount which improves the utilization of carbohydrate, especially starch, in the rumen without unduly stimulating the growth of the potentially harmful bacteria. The ruminant animal need not also be supplied with exogenous fibrolytic enzymes such as xylanase and/or cellulase in the diet.

In the specifically exemplified embodiment using an alpha amylase preparation from Aspergillus oryzae, the dose is from about 2000 to about 20,000 units of activity per bovine per day, desirably from about 4000 to about 16,000 and preferably from about 5000 to about 12,000 units per animal per day. This level of supplementation does not result in lactic acidosis in the rumen, nor does it result in a bloom of lactic acid producing bacteria.

It is a further aspect of the present invention to improve nutrient utilization in the rumen without stimulating the growth of Gram-negative bacteria, thus avoiding an increase in endotoxin in the rumen of the animal to which the relatively low level amylase supplements are included in a grain-containing diet.

It is another aspect of the present invention to use additional feed supplements besides the amylase supplements. For example, dried yeast (Saccharomyces cerevisiae) helps prevent increases in ruminal lactic acid concentration and a concomitant drop in rumen pH. An exemplary yeast product is Yea-sacc (Alltech, Inc., Nicholasville, Ky.). Advantageously, the supplementation rate for this yeast product is about 10 g per cow per day. A specifically exemplified amylase product useful in the practice of any aspect of the present invention is ValidaseFAA Concentrate (food grade amylase, devoid of cellulase and xylanase activity, produced by fermentation of Aspergillus oryzae, sold by Valley Research, Inc., South Bend, Ill.). The ruminant animal could receive, in addition to the amylase supplement as described herein, additional supplementation with an ionophore (e.g., monensin) to help prevent lactic acid from accumulating in the rumen to deleterious levels. A buffering agent such as bicarbonate can also be incorporated into the supplement regime to further insure that the lactic acid concentration and pH in the rumen will remain within the appropriate range.

Still another aspect of the present invention is a diluted enzyme composition comprising exogenous amylase and a carrier, wherein the amylase is present in the composition at a ratio of about 850 to about 9000 FAU units per gram of carrier. Desirably the composition is a dry composition. The carrier can be a biologically inert material such as clay, a mineral supplement suitable for consumption by the ruminant animal, an edible composition such as a dried fermentation extract which is itself substantially devoid of enzymatic activity or dried beet pulp. It is understood that if a concentrated enzyme preparation is to be mixed into a feed such as a grain-based cattle feed, it is desirably diluted with thorough mixing with a material to facilitate subsequent thorough mixing with the animal feed. Assuming each cow or other bovine receives and ingests about 40 kg feed per day, then 250 g of (diluted) enzyme composition per ton of feed (as fed feed, including moisture) is mixed. Typically, on a dry matter basis (DM, dry weight feed) a lactating cow will consume about 20 kg feed per day.

### DETAILED DESCRIPTION OF THE INVENTION

As used herein, an amylase is an enzyme which degrades starch. One enzyme protein may hydrolyze both alpha 1,4 and alpha 1,6 linkages within the starch molecule or there may be separate amylases which hydrolyze these bonds. There are a number of commercially available enzyme preparations of a quality and nature which permits feeding to animals. It is understood that for use in the feed supplementation methods of the present invention the amylase(s) must be active under the conditions of temperature (about 39° C.), pH (about 5.2 to about 6.8) and ionic strength in the rumen. In the context of the present disclosure, a unit of amylase activity is as given in Example 2 herein below.

For supplementation of a ruminant animal starch-containing diet, a source of amylase is desirably formulated together with a carrier suitable for consumption by the animal, and optionally additional ingredients to improve the ease of use, such as flow control agents. The source of the enzyme can be an extract (or fermentation extract) derived from an amylase-producing organism, including but not limited to Aspergilus oryzae. The carrier can be a mineral supplement suitable for the animal, ground grain or roughage, or it can be a dried fermentation soluble preparation, for example, the results of drying spent medium from a yeast fermentation after the removal of solids.

In order to examine the effect of readily fermentable starch on ruminal VFA concentrations in dairy cows, a representative TMR (Table 1) was supplemented with 1 kg dry ground corn at the time of feeding. Non-significant numerical increases in the concentrations of acetate, propionate and butyrate were observed across a 4 h period after feeding when supplemental corn was added to the diet. However, there was an increase in the proportion of butyrate and a decrease of the proportion of propionate within the VFA compound class in the rumen.

TABLE 1
Total Mixed Ration Composition

| Component | As is basis | DM basis |
|---|---|---|
| Dry matter, % | 62.00 | |
| Moisture | 38.00 | |
| Protein, % | 11.28 | 18.20 |
| Acid detergent fiber, % | 11.22 | 18.10 |
| Neutral detergent fiber, % | 17.42 | 28.10 |
| Crude fiber, % | 8.43 | 13.60 |
| Calcium, % | 0.77 | 1.24 |
| Phosphorous, % | 0.25 | 0.40 |
| Potassium, % | 0.86 | 1.39 |
| Magnesium, % | 0.19 | 0.31 |
| Sodium, % | 0.12 | 0.19 |
| TDN (estimated), % | 49.22 | 79.39 |
| NE₁, Mcal/kg | 0.99 | 1.60 |
| NE_{g} Mcal/kg | 0.89 | 1.43 |
| MEₘ, Mcal/kg | 1.30 | 2.10 |
| Fat, % | 1.92 | 3.09 |
| Starch, % | 16.80 | 27.10 |

To explore the effect of making the hexoses of starch more available to the rumen microbiota, amylase was added to the TMR. This addition enhanced the in situ disappearance of starch during the initial 6 h period without altering the in situ disappearance of dietary neutral detergent fiber (NDF). The effects of supplemental amylase on starch disappearance were not reflected in significant changes in ruminal VFA concentrations at the amylase supplementation levels tested.

Twenty intact and four ruminally fistulated lactating Holstein cows were used in a 4×4 latin square design, replicated six times, to examine the effects of four concentrations of a supplemental enzyme preparation on milk production, milk composition, and ruminal digestibility and fermentation. The cows were assigned to one of six squares based on DIM and presence or absence of ruminal fistulas. The treatments included enzyme supplementation at 0, 6000, 12,000 and 18,000 units fungal alpha amylase per cow per day (Validase FAA). Treatment periods included a 14 day adaptation period prior to a 7 day collection period. Enzyme supplementation had a quadratic effect on milk production (P=0.02). The maximum milk yield was obtained with 6000 units fungal alpha-amylase per cow per day. Percent fat and protein in milk were increased in the presence of enzyme supplement and therefore resulted in greater total fat and protein in milk. Enzyme supplementation had a significant cubic effect (P=0.03) on milk urea nitrogen (MUN). The addition of 6000 units fungal alpha amylase per cow per d resulted in lower MUN than in any other treatment. Enzyme addition did not affect ruminal starch or NDF digestibility of corn silage but increased (P<0.01) the ruminal starch digestibility of grain corn after in situ incubation for 24 h by 7.8%. The addition of 6000 units fungal alpha amylase per cow per d did not affect total VFA concentrations in the rumen but reduced (P<0.01) propionate and increased (P<0.01) acetate and butyrate proportions by 9.1, 1.5 and 9.3%, respectively. These results indicate that low concentrations of enzyme enhance performance in ruminant animals by modifiying fermentation in the rumen without significant increases in digestibility.

In addition to beneficial affects on the microbial fatty acid composition of the rumen, there was an increase in milk production due to the supplementation with the relatively low levels of amylase described herein. There was also an increase in the fat and protein contents of the milk produced with the amylase supplementation. In addition, there was a decrease in the amount of urea in that milk as well, thus further improving the quality of the milk.

The economics of beef production can also be improved by supplementing starch-containing feed with at least one amylase at levels as described herein. Nutrient utilization and weight gain are improved by the amylase supplementation in the absence of feeding greater amounts of feed.

Without wishing to be bound by any particular theory, the present inventors believe that addition of the relatively low levels of amylase to the ruminant diet (including sources of starch) results in a stimulation of the growth of beneficial rumen bacteria by making the hexose in starch more available in the rumen without creating such a high level of dextrins and/or glucose that there is a significant increase in lactic acid production or a significant decrease in rumen pH. Desirably the rumen pH remains between about 5.8 and 6.4.

A number of amylase-containing preparations are commercially available. The amylase (or combination of amylase activities) must have activity in the conditions of the rumen—pH from about 5.2 to about 6.8 and temperature of about 39° C., and desirably, the enzyme has activity between about 33 and 45° C. Enzyme activities can be measured by a number of assay methods, but for comparison to the present disclosure, it is recommended that measurements are carried out as described herein.

For animal dietary supplementation, the amylase-containing material is provided to the ruminant animals, conveniently by addition to and mixing with the feed rations or by providing the enzyme supplement at the same time as the feed rations are provided. The daily dose recommended herein can be provided as one administration per day, or the daily dose can be provided more than once during the day. Often with dairy cattle, there are two or three feedings per day in addition to the offering of hay or other fibrous feed at other times. The amylase(s) can be added to the diet in the form of a dry material, or the enzyme can be administered in the form of a liquid formulation which is sprayed on the feed. It is well understood in the art how to formulate enzyme preparations for good shelf life and for ease of use.

TABLE 2
Effects of various supplemental enzyme concentrations on milk
production and composition in lactating Holstein cows.
Enzyme supplement (amylase units * cow⁻¹ * d⁻¹)

| Item | 0 | 6 000 | 12 000 | 18 000 | SEM | P-value |
|---|---|---|---|---|---|---|
| Milk yield, kg * d⁻¹ | 29.2 | 30.7 | 30.4 | 29.6 | 0.49 | .1269 |
| 3.5% FCM, kg * d⁻¹ | 30.2 | 32.0 | 31.6 | 30.7 | 0.54 | .0798 |
| Milk fat, kg * d⁻¹ | 1.08 | 1.15 | 1.14 | 1.10 | 0.02 | .0960 |
| Milk protein, kg * d⁻¹ | 0.99 | 1.04 | 1.03 | 1.02 | 0.02 | .1938 |
| Fat % | 3.71 | 3.78 | 3.78 | 3.75 | 0.04 | .5762 |
| Protein % | 3.43 | 3.43 | 3.43 | 3.46 | 0.02 | .7268 |
| MUN, mg * dl⁻¹ | 8.84 | 8.16 | 8.85 | 8.80 | 0.21 | .0637 |
| Milk allantoin, | 155.6 | 156.1 | 152.6 | 157.7 | 1.75 | .2297 |
| mg * l⁻¹ | | | | | | |

TABLE 3
Effects of various supplemental enzyme concentrations on ruminal
VFA and ammonia concentrations in lactating Holstein cows.
Enzyme supplement (amylase units * cow⁻¹ * d⁻¹)

| Item | 0 | 6 000 | 12 000 | 18 000 | SEM | P-value |
|---|---|---|---|---|---|---|
| Total VFA, mM | 164.9 | 163.1 | 156.9 | 159.3 | 1.89 | .4545 |
| VFA, mol * 100 mol⁻¹ | | | | | | |
| Acetate | 60.7 | 61.6 | 63.2 | 62.0 | 0.15 | .1096 |
| Propionate | 21.9 | 19.9 | 18.7 | 19.8 | 0.16 | .1323 |
| Butyrate | 12.9 | 14.1 | 13.9 | 13.9 | 0.12 | .1957 |
| A: P | 2.85 | 3.15 | 3.42 | 3.17 | 0.57 | .0983 |
| NH₃, mM | 5.94 | 6.06 | 5.78 | 6.01 | 0.54 | .9870 |

All references cited in the present application are incorporated by reference herein to the extent that there is no inconsistency with the present disclosure.

The examples and descriptions are provided herein for illustrative purposes, and are not intended to limit the scope of the invention as claimed herein. Any variations in the exemplified articles which occur to the skilled artisan are intended to fall within the scope of the present invention.

### EXAMPLES

### Example 1

### Animal Husbandry

In the experiments described herein, lactating Holstein cows are housed and fed in accordance with current accepted dairy practice. They are fed twice a day approximately 20 kg DM of a typical TMR and 3.5 kg of hay per day. The TMR (total mixed ration) nutrient composition is given in Table 1. Supplementation with amylase was as given in Tables 2 and 3.

### Example 2

### Assay of Amylase Activity

Alpha amylases (IUB #3.2.1.1) break down the alpha 1,4 glucosidic linkages of dextrin to yield maltose and smaller dextrins. The breakdown products are reacted with an iodine solution and the color produced is compared to a standard color solution. As starch is broken down the color changes from blue to red-brown. One FAU unit is the amount of enzyme which will dextrinize soluble starch at the rate of 1 g per hour at 30° C. and pH 4.8.

Equipment needed includes a spectrophotometer for measuring absorbance at 617 nm, a 30° C. water bath and a timer.

2M Acetate buffer is prepare by dissolving 164 g of anhydrous sodium acetate in about 500 mL of distilled water. 120 mL of glacial acetic acid is added, and the pH is adjusted to 4.8 with glacial acetic acid. This mixture is diluted to 1 L with distilled water and mixed.

A buffered starch solution is prepared by dispersing 2.0 g of potato soluble starch (Sigma Chemical Co., St. Louis, Mo., #2630) in 20 mL of distilled water and pouring slowly into 600 mL of boiling water. This mixture is boiled with stirring for 1-2 minutes and then quantitatively transferred to a 1 L volumetric flask with the aid of water. 5 mL of Acetate buffer pH 4.8 is added, and the mixture is diluted and mixed to volume with water. This mixture is prepared fresh daily.

The enzyme dilution solution is prepared as follows: In a 1 L volumetric flask, 0.585 g sodium chloride and 2.22 g calcium chloride are added to 800 mL distilled water. 20 mL of 2 M acetate buffer is added and the pH is adjusted to 4.8 with 1 M NaOH, and the volume is adjusted to 1 L with distilled water.

The stock iodine solution is prepared by dissolving 1.1 g iodine and 2.2 g potassium iodide in 25 mL distilled water, transferring to a 50 mL volumetric flask and filling to volume. The solution is stored in darkness, and a fresh solution is made monthly. The working iodine solution is prepared by dissolving 10 g of potassium iodide in 200 mL distilled water; 1.0 mL of stock iodine solution is added, and the volume is adjusted to 250 mL with distilled water. This working iodine solution is prepared fresh daily.

The enzyme samples are diluted in enzyme dilution solution so as to give an end point between 10 to 20 minutes in the procedure as described below.

For each sample to be analyzed, 5 mL of buffered starch solution is placed in a 20 mm×150 mm test tube and allowed to equilibrate in a 30° C. water bath for 5-10 minutes. For each enzyme sample to be analyzed, 5 mL of the working iodine solution is dispensed into 5-15 separate tubes, and the tubes are placed in the 30° C. water bath. The diluted enzyme solution is placed in a 30° C. water bath.

The spectrophotometer (617 nm) is zeroed using distilled water, and the absorbance in each tube is measured and recorded. The absorbance at 617 nm of the standard color solution should be about 0.410.

A 2.5 mL aliquot of the enzyme solution is transferred into the starch flask and mixed. The reactions is allowed to proceed (and is timed) at 30° C. After 9-10 minutes of incubation, and at definite time intervals thereafter, 1 mL aliquots of the reaction mixture are placed into 5 mL aliquots of the working iodine solution, mixed and the absorbance is determined. As the O.D. of the reaction mixture approaches that of the color standard, the absorbence is measured every 30 seconds. Starch hydrolysis is determined by referring to a color standard or regression or standard curve encompassing the data point.

The units of amylase activity are calculated as follows:

$$FAU / g = \frac{40 \times F}{T}$$

40=is a constant derived from the 400 mg of starch (20 mL of a 2% solution)

T=Time of reaction in minutes

F=Dilution Factor for the enzyme (1000).

### Example 3

### Determination of Volatile Fatty Acids

Samples containing VFA were taken from the rumen of the fistulated animals at certain times after feeding with the amylase-supplemented feeds or the non-supplemented controls. Volatile fatty acid concentrations were determined by gas chromatography [Erwin et al. (1961) J. Dairy Sci. 44, 1768-1771]. Samples were collected and frozen until analysis. A 1 mL aliquot from each sample was clarified by centrifugation with 0.2 mL of 25% metaphosphoric acid. Supernatant from each sample (1 μl) was injected on a Hewlett-Packard model 5890 series II gas chromatograph equipped with a 6 ft×4 mm glass column packed with 10% SP-1000/1% H₃PO₄ on 100/120 Chromosorb WAW (Supelco Inc., Bellefonte, Pa.). The carrier gas flow rate was maintained at 32 mL/min and the oven temperature was held constant at 135° C. Volatile fatty acids produced by each culture were determined by subtracting the average VFA concentrations at 0 h from the VFA concentrations after in vitro incubation. Hexose utilization was estimated stoichiometrically from VFA production by calculating the theoretical fermentation balance [Wolin (1960)].

Data were analyzed by the general linear model procedure of Statistical Analysis Software (SAS Institute, Cary, N.C.) as a one-way treatment classification in a replicated Latin square design (cows=columns, periods=rows). Squares were considered to be fixed effects. Periods and treatments were assumed to be independent and cows were nested within square. Orthogonal polynomials were used to partition linear, quadratic and cubic effects of enzyme supplementation.

### Example 4

### Influence of AMAIZE™ Supplementation on Milk Production and Milk Composition

A series of field trials was conducted in eight commercial dairy herds. Five herds were located in Ontario, and three herds were located in Pennsylvania. AMAIZE™ (Alltech, Inc., Nicholasville, Ky., amylase-containing enzyme nutritional supplement) was added to the base herd ration at the rate of 12 grams/head/day following an initial Dairy Herd Improvement (DHI) test (control). The enzyme was fed for approximately 30 days until the next DHI test (Amylase). Summaries of the herds and diets utilized in the trial are presented in Tables 4 and 5. Forage:concentrate ratios ranged from 44:56 to 59:41. Forage sources ranged from all alfalfa haylage to greater than 90% corn silage (% forage DM). Grains fed included dry corn, high moisture corn, ear corn, and ground barley. Data were analyzed for statistical significance by a Student's t-test (Amylase—Control, null hypothesis: difference=0, alternate hypothesis: difference>0).

Average milk production and composition data are shown in Table 6. Supplementation of AMAIZE™ resulted in an average increase of 2.9 lbs of milk per cow per day (P=0.187) and 3.7 lbs of 3.5% fat-corrected milk per cow per day (P=0.0076). Milk production and fat-corrected milk improved in 7 of 8 herds when fed AMAIZE™. Average milk fat percentage increased from 3.90 to 3.95 (P=0.0227) but effects of AMAIZE™ on fat percentage were less consistent with 5 of 8 herds reporting higher milk fat percentages. Milk fat yield was improved with AMAIZE™ inclusion in 4 of 8 herds. Average milk protein percentage was higher when cows were fed AMAIZE™ (3.26 versus 3.33, P=0.001) and was improved in 7 of 8 herds. Milk protein yields were higher in 7 of 8 herds on AMAIZE™.

Supplementation of AMAIZE™ improved average milk production, fat-corrected milk production, milk fat yield, and milk protein yield across the eight herds in this trial. Improvements in milk yield, 3.5% fat-corrected milk yield, and milk protein percentage, and milk protein yield were observed in 7 of 8 herds. The response in milk fat percentage and milk fat yield to AMAIZE™ was less consistent, but it is interesting to note that the milk fat yield was either improved or equal in all herds when cows were fed AMAIZE™.

In this trial, the response to amylase supplementation did not appear to be dependent upon the rations fed. Without wishing to be bound by theory, it is believed that improvements in performance of cows fed amylase supplements are due to changes in ruminal fermentation of starch, resulting in more energy available to the cows.

TABLE 4
Summary of animals utilized in field trials

| | |
|---|---|
| Number of Cows | Days in Milk |

| Farm | Control | Amylase | Control | Amylase |
|---|---|---|---|---|
| 1 | 51 | 53 | 170 | 177 |
| 2 | 30 | 31 | 160 | 177 |
| 3 | 124 | 131 | 182 | 186 |
| 4 | 60 | 67 | 140 | 170 |
| 5 | 70 | 70 | NA | NA |
| 6 | 80 | 80 | 161 | 157 |
| 7 | 65 | 65 | NA | NA |
| 8 | 60 | 60 | 162 | 168 |

NA = Not available

TABLE 5
Summary of diets utilized in field trials.

| | F:C | Forage | | Grain | |
|---|---|---|---|---|---|
| Farm | Ratio | Primary | Secondary | Primary | Secondary |
| 1 | 47:53 | Haylage | Corn silage | Dry corn | Barley |
| 2 | 52:48 | Haylage | Corn silage | HM corn | Barley |
| 3 | 48:52 | Haylage | Corn silage | HM corn | Barley |
| 4 | 55:45 | Corn silage | Haylage | HM corn | Wheat bran |
| 5 | 44:56 | Corn silage | Haylage | Ear corn | Dry corn |
| 6 | 53:47 | Corn silage | Haylage | Hm corn | |
| 7 | 59:41 | Corn silage | Haylage | Dry corn | |
| 8 | 48:52 | Corn silage | Haylage | HM corn | Barley |

TABLE 6
Effect of amylase supplemention on milk
production and composition

| | Control | Amylase | P-value |
|---|---|---|---|
| Days in milk | 163 | 173 | |
| Milk yield (lbs) | 68.8 | 71.7 | 0.0187 |
| 3.5% FCM (lbs) | 73.2 | 76.9 | 0.0076 |
| Milk fat (lbs) | 2.68 | 2.83 | 0.0227 |
| Milk fat (%) | 3.90 | 3.95 | |
| Milk protein (lbs) | 2.25 | 2.39 | 0.0010 |
| Milk protein (%) | 3.26 | 3.33 | |

### Example 5

### Effect of Amylase Supplementation on Milk Production—Paired Cow Study

A commercial dairy herd in Ontario was utilized for this short-term trial. Fifty-four lactating cows were paired based on milk production and DIM and split into two groups. The control group received the base ration and the AMAIZE™ group received the base ration plus 12 gram/head/day of the supplement. Milk production was measured individually prior to and 3 days after addition of the amylase dietary supplement. Data were analyzed for statistical significance by a paired Student's t-test (Amylase—Control, null hypothesis: difference=0, alternate hypothesis: difference>0).

Group milk production is shown in Table 7. On day 0, the difference in milk production between the control and amylase groups was 2.78 lbs per cow per day. After 3 days on the maylaes supplement, the difference in milk yield was 8.09 lbs (P=0.023).

Supplementation with amylase resulted in a greater than 5 lb improvement in daily milk yield in 3 days. Milk production responses to amylase supplementation at the levels taught herein can be demonstrated in as little as 3 days on the supplement.

TABLE 7
Effects of amylase supplementation on milk
production (paired field trial)

| | Amylase-Control | | |
|---|---|---|---|
| Day | Milk yield (lbs/cow/d) | SE | P-value¹ |
| 0 | 2.78 | 4.06 | 0.250 |
| 3 | 8.09 | 3.84 | 0.023 |

¹Probability that null hypothesis (Amylase−Control = 0) is true.

### Example 6

### Effect of Amylase Supplementation on Milk Production and Body Condition

A commercial dairy herd in Ontario was utilized for this trial. Amylase nutitional supplement (AMAIZE™, Alltech, Inc., Nicholasville, Ky.) was added to the ration at 12 grams/head/day beginning in early spring. Milk production and body condition score were available for 47 of 51 cows and was compared on two dates, prior to amylase supplementation and approximately 1 month after beginning amylase supplementation. Data were not analyzed for statistical significance.

Herd milk production and body condition score is shown in Table 8. After approximately 7 weeks on the amylase supplement, the milk production of 84.0 lbs/day was nearly identical to the starting milk yield of 83.6 lbs. Based on an expected decline in milk production of 8% per month (or 2.67%/day), milk production for these 47 cows at 199 days into milk (DIM) was predicted to have been at 75 lbs/cow. Average body condition score was greater after 7 weeks on supplementation with amylase.

Feeding of the amylase supplement to this herd of dairy cows appeared to help cows hold milk production despite increasing days in milk. Cows were also able to gain condition while holding milk production.

TABLE 8
Effects of amylase supplementation on milk
production and body condition

| | | Milk | Expected¹ | |
|---|---|---|---|---|
| | | Yield | Milk Yield | |
| | DIM | (lbs/day) | (lbs/day) | BCS |
| Before | 160 | 83.6 | | 3.44 |
| amylaase | | | | |
| Mar. 12, 2002 | | | | |
| After amylase | 199 | 84.0 | 75.0 | 3.52 |
| Apr. 20, 2002 | | | | |

¹Predicted milk = previous milk yield adjusted for change in DIM

### Example 7

### Amylase Supplementation Field Trials—Southern States

Whole herd field trials were conducted in summer in 10 commercial dairy herds (approximately 1600 cows) in Virginia and Kentucky. Nine herds started feeding amylase supplement (AMAIZE™) (12 grams/head/day) in early summer while one dairy began feeding the product in mid summer. Monthly DHI test data and other calculated data (Fat Corrected Milk, FCM; Energy Corrected Milk, ECM) was summarized by month relative to Amaize addition. Data were not analyzed for statistical significance.

Milk production and composition are presented in Table 9. DHI tests were available for all 10 herds for two months prior to and two months following amylase supplement addition. Milk production was numerically lower while cows were supplemented with amylase, but milk components were similar.

Results of these field trials were likely influenced by heat stress. However, the supplementation of the diet with amylase is believed to have helped maintain milk fat percentage, typically lower during heat stress.

TABLE 9
Effects of Amylase Supplementation on Milk Production

| | Amylase “Status” | | | | | | | |
|---|---|---|---|---|---|---|---|---|
| | Pre − 2 | Pre − 1 | Month 1 | Month 2 | Month 3 | Month 4 | Post + 1 | Post + 2 |
| | Off | Off | On | On | On | On | Off | Off |
| Total Test Dates | 10 | 10 | 10 | 10 | 3 | 1 | 8 | 4 |
| Days on/off | −45 | −12 | 20 | 55 | 74 | 93 | 23 | 54 |
| Amylase | | | | | | | | |
| # of cows | 1569 | 1610 | 1610 | 1553 | 395 | 195 | 1157 | 719 |
| Days in milk | 193 | 200 | 203 | 205 | 196 | 198 | 201 | 196 |
| Milk yield (lbs) | 72.9 | 68.4 | 64.9 | 62.3 | 70.1 | 62.7 | 63.2 | 62.6 |
| Milk fat (%) | 3.64% | 3.64% | 3.65% | 3.66% | 3.80% | 4.40% | 3.73% | 3.73% |
| Milk protein (%) | 2.98% | 2.93% | 2.90% | 2.96% | 3.04% | 3.20% | 3.02% | 3.09% |
| Milk fat (lbs) | 2.65 | 2.49 | 2.37 | 2.28 | 2.64 | 2.76 | 2.35 | 2.34 |
| Milk protein (lbs) | 2.17 | 2.00 | 1.88 | 1.85 | 2.13 | 2.01 | 1.90 | 1.93 |
| 150-day milk (lbs) | 78.0 | 74.6 | 72.7 | 70.8 | 78.4 | 69.7 | 71.7 | 71.6 |
| 3.5% FCM (lbs) | 74.5 | 70.0 | 66.4 | 63.9 | 73.1 | 71.9 | 65.4 | 64.9 |
| ECM (lbs) | 73.0 | 68.4 | 64.7 | 62.6 | 71.7 | 70.0 | 64.1 | 63.9 |

### Example 8

### Performance Study with Feedlot Cattle

The effects of roughage source and amylase supplementation on the performance and carcass characteristics of finishing beef steers were examined as described below.

One hundred sixty-two steers (mixed breeding—British and British x Continental) were received at a Texas facility in spring. The average body weight (BW) of the group on arrival was 753 lb. The cattle were housed in 12 soil-surfaced pens (13 to 14 steers per pen) and offered 9 lb per steer of a 70% concentrate diet. The following day, all steers were processed and returned to the same soil-surfaced pens to which they had been randomly allotted on the previous day. Approximately two weeks later, all cattle were switched to an 80% concentrate diet. Feed delivery to each pen was 95% of the delivery for the previous day.

All cattle were weighed to obtain a BW for sorting into blocks and treatment groups. One week later, the 120 steers selected for the experiment were brought through the working chute, where they were weighed and implanted with Ralgro (Schering-Plough Animal Health, Union, N.J., ear implant containing 36 mg zeranol) in their right ear and sorted to their assigned pens. After sorting to pens, the cattle were fed the same 90% concentrate diet they had received previously.

Four dietary treatments, arranged in a 2×2 factorial, were used in a randomized complete block design. Pen was the experimental unit (six pens per treatment with five steers per pen for a total of 120 steers). All diets contained 90% concentrate and the four treatments were as follows:

ALF−: Alfalfa as the roughage source, with no added amylase.

ALF+: Alfalfa as the roughage source plus amylase.

CSH−: Cottonseed hulls as the roughage source with no added amylase.

CSH+: Cottonseed hulls as the roughage source plus amylase.

Amylase was added as a premix. The premix was 46.07% ground corn (DM basis) and 53.93% amylase supplement (DM basis). The supplement contains amylase-containing extract produced by Aspergillus oryzae, and dried fermentation solubles (spent medium from a Saccharomyces cerevisiae fermentation was dried after the removal of solids). The enzymatic activity was 1395 units per pound of premix.

Ingredient composition of the diets fed during the experiment is shown in Table 20. Each diet contained the same intermediate premix which supplied protein, various minerals and vitamins, Rumensin (monensin sodium, Elanco, Indianapolis, Ind., 30 g/ton, DM basis), and Tylan (tylosin, 8 g/ton, DM basis).

Standard procedures for feeding and weighing were used throughout the experiment. Mixing and feeding order of treatment diets throughout the experiment was CSH−, ALF−, CSH+ and ALF+. Dry matter (DM) determinations on ingredients used in the experimental diets were made every 2 wk throughout the experiment. These DM values were used to calculate the DM content of each dietary ingredient during the experiment. In addition, samples of mixed feed delivered to feed bunks were taken weekly throughout the experiment. These bunk sample DM values were used to compute average DM intake (DMI) by the cattle in each pen. Samples of feed taken from the bunk were composited for each period of the experiment. Composited samples were ground to pass a 2-mm screen in a Wiley mill and analyzed for DM, ash, CP, acid detergent fiber, Ca, and P using AOAC (Official Methods of Analysis, 15^{th} ed., 1990) procedures.

Each feed bunk of the 24 pens was evaluated visually at approximately 0700 to 0730 daily. The quantity of feed remaining in each bunk was estimated, and the suggested daily allotment of feed for each pen was recorded. This bunk-reading process was designed to allow for little or no accumulation of unconsumed feed (0 to 1 lb per pen). A challenge process was to ensure that the cattle were consuming the maximum quantity of feed possible. Feed bunks were cleaned, and unconsumed feed was weighed at intervals (corresponding to intermediate weigh dates) throughout the trial and DM content of these bunk weighback samples was determined. Bunk weighbacks and DM determinations were used to calculate DMI by each pen.

After 28, 84, and 112 d on feed, cattle were weighed on a pen basis using a platform scale (+5 lb). On d 56 and just before shipment to slaughter, BW measurements were obtained for individual animal basis using a single-animal scale (C & S Single-Animal Squeeze Chute set on four load cells). On d 112, it was visually estimated that steers in Blocks 5 and 6 had sufficient finish to grade USDA Choice in approximately 2 wk; therefore, steers were scheduled to ship to slaughter on d 133 of the experiment. On d 140 the remainder of the cattle were weighed (pen basis) for the regularly scheduled weigh day. Steers in Blocks 3 and 4 were weighed individually, and shipped to slaughter on d 154 of the experiment. Steers in Blocks 1 and 2 were weighed individually on d 168 and were shipped to slaughter.

Carcass data were collected by trained personnel; evaluations were according to standard protocols. Data included hot carcass weight, fat thickness at the 12^{th} rib, longissimus muscle area, percentage of kidney, pelvic and heart fat, liver score, marbling score, quality grade, and yield grade.

Performance data and carcass data were analyzed as a randomized complete block with a 2×2 factorial arrangement of treatments. The fixed effects of the model included roughage source, Amaize addition, and the interaction of roughage source x Amaize addition. Block was the random effect. Data were analyzed using PROC MIXED of SAS (SAS Inst. Inc., Cary, N.C.). Percentage of carcasses grading USDA Choice were analyzed using a non-parametric model (PROC CATMOD of SAS).

Performance data are presented in Table 11. An amylase x roughage source interaction was detected for d 0 to 28 ADG (P=0.02). Cattle fed CSH+ had greater ADG than those fed ALF+, ALF−, or those fed CSH−. Similarly, for ADG from d 0 to 112, an Amaize x roughage source interaction was observed (P=0.04); cattle fed CSH+had greater gains than those fed CSH−. However, for d 0 to 56, d 0 to 84, and overall ADG, no effects (P>0.10) of roughage source, amylase, or the interaction of roughage source x amylase were detected.

An amylase x roughage source interaction (P<0.10) was observed for DMI on d 0 to 56 and on d 0 to 112. Cattle fed the CSH+had greater DMI than in the other three treatments. No differences (P>0.10) were noted among treatments for feed efficiency at any period during the finishing phase. Although differences were not significant, there was a strong trend for a roughage source x Amaize interaction for overall ADG (P<0.12), DMI (P<0.20), and feed:gain (P<0.18). This trend was largely the result of the increased ADG and DMI, and improved feed:gain when amylase was supplemented to cattle fed cottonseed hulls as the roughage source. It is unclear, however, why cattle fed the amylase supplement and cottonseed hulls had greater DMI and ADG at various periods of the study. Perhaps differences in ruminal digesta kinetics between alfalfa and cottonseed hulls affected the need for supplemental amylase either in the rumen or intestines. Additionally, diets containing cottonseed hulls had a higher concentration of cottonseed meal. Perhaps different protein sources alter the need for additional amylase or the effect of amylase on ruminal fermentation.

Roughage source did not affect DMI, ADG, or feed efficiency at any point in the feeding period. Based upon these data, it seems that performance is not affected by roughage source when the percentage of NDF supplied by the roughage source is similar.

Neither roughage source, amylase addition nor the interaction affected (P>0.10) carcass weight, dressing percent, fat thickness, percentage of kidney, pelvic, and heart fat, marbling score, yield grade, or percentage of cattle grading USDA Choice or better. The addition of amylase increased longissimus muscle area (P=0.05), but the mechanism is unknown. No effect (P>0.10) of amylase, roughage, or amylase x roughage was noted for liver score data.

TABLE 10
Ingredient composition (%, DM basis) of the experimental diets

| | Treatments^{a} | | | |
|---|---|---|---|---|
| Ingredient | ALF+ | ALF− | CSH+ | SH− |
| Alfalfa hay, mid-bloom | 12.13 | 12.13 | — | — |
| Cottonseed hulls | — | — | 6.53 | 6.53 |
| Steam flaked corn | 75.66 | 75.67 | 79.47 | 79.46 |
| Cane molasses | 4.14 | 4.14 | 4.14 | 4.14 |
| Tallow | 3.05 | 3.05 | 3.05 | 3.05 |
| Urea | 0.92 | 0.92 | 1.22 | 1.22 |
| Cottonseed meal | 1.30 | 1.30 | 2.52 | 2.52 |
| TTU 2.5 supplement | 2.54 | 2.54 | 2.54 | 2.56 |
| Limestone | — | — | 0.27 | 0.27 |
| Control premix^{b} | — | 0.25 | — | 0.25 |
| Amylase premix^{c} | 0.26 | — | 0.26 | — |

^{a}Dietary treatments: ALF+ = alfalfa as the roughage source plus amylase; ALF− = alfalfa as the roughage source without Amaize; CSH+ = cottonseed hulls as the roughage source plus Amylase; CSH− = cottonseed hulls as the roughage source without Amylase.
^{b}Control premix was composed of ground corn only.
^{c}Amylase premix was composed (DM basis) of ground corn (46.07%) and Amylase supplement (53.93%).

TABLE 11
Effects of roughage source and the addition of amylase on
feedlot performance by finishing beef steers^{a,b}

| | ALF | | CSH | | | Effect | | |
|---|---|---|---|---|---|---|---|---|
| Item | + | − | + | − | SE^{d} | A | R | A × R |
| Final BW, lb | 1258.9 | 1,272.1 | 1282.9 | 1,250.2 | 26.64 | 0.55 | 0.95 | 0.17 |
| ADG, lb | | | | | | | | |
| d 0 to 28 | 3.79 | 3.85 | 4.14 | 3.60 | 0.16 | 0.06 | 0.66 | 0.02 |
| d 0 to 56 | 3.19 | 3.29 | 3.35 | 3.10 | 0.14 | 0.47 | 0.87 | 0.10 |
| d 0 to 84 | 3.26 | 3.37 | 3.44 | 3.20 | 0.11 | 0.58 | 0.98 | 0.15 |
| d 0 to 112 | 3.16 | 3.23 | 3.37 | 3.05 | 0.09 | 0.19 | 0.87 | 0.04 |
| DMI, Ins | | | | | | | | |
| d 0 to 28 | 16.60 | 16.06 | 17.28 | 16.60 | 0.53 | 0.10 | 0.10 | 0.85 |
| d 0 to 56 | 16.37 | 16.41 | 17.26 | 16.42 | 0.48 | 0.12 | 0.09 | 0.09 |
| d 0 to 84 | 16.35 | 16.31 | 17.18 | 16.31 | 0.45 | 0.09 | 0.11 | 0.11 |
| d 0 to 112 | 16.47 | 16.51 | 17.33 | 16.36 | 0.43 | 0.07 | 0.16 | 0.05 |
| Feed:gain | | | | | | | | |
| d 0 to 28 | 4.40 | 4.18 | 4,205.1 | 4.63 | 0.13 | 0.43 | 0.38 | 0.02 |
| d 0 to 56 | 5.16 | 5.01 | 8 | 5.34 | 0.18 | 0.99 | 0.22 | 0.30 |
| d 0 to 84 | 5.03 | 4.85 | 5.00 | 5.11 | 0.12 | 0.75 | 0.34 | 0.25 |
| d 0 to 112 | 5.22 | 5.11 | 5.15 | 5.37 | 0.10 | 0.60 | 0.36 | 0.13 |

^{a}Roughage source: ALF = alfalfa hay; CSH = cottonseed hulls.
^{b}Amylase addition: + = added Amylase to the diet; − = no added Amylase to the diet.
^{c}Observed significance level for the effect: A = Amylase effect; R = roughage effect; A × R = Amylase × roughage interaction.
^{d}Standard error of the treatment means, n = six pens per treatment.
^{e}Adjusted final BW = hot carcass weight divided by a common dressing percent of 62.84%.

### Example 9

### In vitro Studies with Pure Cultures of Ruminal Microorganisms

A series of experiments were performed to examine the effects of an exogenous enzyme preparation containing amylase activity on the growth characteristics of representative rumen bacteria. Pure cultures of Butyrivibrio fibrisolvens strains D1 49, and A38, Streptococcus bovis strain S1, Megasphaera elsdenii strain T81, and Selenomonas ruminantium strain GA192 were grown anaerobically on medium 10 broth containing soluble potato starch (1.0 g/L) as the sole carbohydrate source. Enzyme treatment was applied immediately prior to bacterial inoculation by adding 0.1 ml of an enzyme solution to provide a final concentration of 0.06 units amylase/ml. Control cultures received 0.1 ml of a solution prepared with fermentation solubles (enzyme carrier). Microbial growth was estimated in each culture by measuring turbidity (600 nm) over time. The addition of supplemental amylase enhanced the growth rates of Butyrivibrio fibrisolvens strain D1, Selenomonas ruminantium strain GA192 and Megasphaera elsdenii strain T81. Supplemental amylase had no effects on the growth rates of Streptococcus bovis strain S1 is and Butyrivibrio fibrisolvens strain 49 and reduced the growth rate of Butyrivibrio fibrisolvens strain A38 (Table 12). Supplemental amylase also enhanced the growth (0.373 vs. 0.493 OD at 15 h; P<0.05) of Butyrivibrio fibrisolvens strain D1 when maltodextrins (1.0 g/L) with an average molecular weight of 3600 and a dextrose equivalence range of 4-7 were included in medium 10 broth as the sole carbohydrate source but did not affect its growth when lower molecular weight maltodextrins were used. Exogenous supplemental amylase enhances the growth of specific strains of ruminal bacteria that do not grow efficiently on starch or high molecular weight maltodextrins.

TABLE 12
Effects of supplemental amylase on the growth
rates of ruminal bacteria on starch

Growth rate (OD/hr)

| Organism | Control | Enzyme | SE |
|---|---|---|---|
| B. fibrisolvens D1^{a} | 0.007 | 0.168 | 0.020 |
| B. fibrisolvens 49 | 0.043 | 0.046 | 0.010 |
| B. fibrisolvens A38^{b} | 0.131 | 0.076 | 0.024 |
| S. ruminantium GA192^{a} | 0.004 | 0.085 | 0.001 |
| M. elsdenii T81^{a} | 0.012 | 0.036 | 0.001 |
| S. bovis S1 | 0.278 | 0.282 | 0.020 |

^{a}Treatment effects (P < 0.05).
^{b}Treatment effects (P < 0.09).

What is claimed is:

1. A method for improving ruminal fermentation efficiency of a starch feed component in a ruminant animal, said method comprising the step of:
orally administering a composition comprising at least one amylase enzyme to a ruminant animal, wherein said ruminant animal is fed a diet comprising starch and wherein said animal receives a dose of at least one amylase of from about 2000 to about 20,000 units of amylase per day.

2. The method of claim 1, wherein the animal receives a dose of the at least one amylase of from about 4000 to about 16,000 units of amylase per day.

3. The method of claim 1, wherein the animal receives a dose of the at least one amylase of from about 5000 to about 10,000 units of amylase per day.

4. The method of claim 1, wherein the ruminant animal is a bovine animal.

5. The method of claim 4, wherein the ruminant animal is a lactating bovine animal.

6. The method of claim 4, wherein the ruminant animal is a feedlot bovine animal.

7. The method of claim 4, wherein the ruminant animal is a pregnant bovine animal.

8. The method of claim 1, wherein the composition administered to the ruminant animal contains no cellulase activity and no xylanase activity.

9. The method of claim 1, wherein the at least one amylase is from Aspergillus oryzae.

10. The method of claim 9, wherein the composition is added to the diet.

11. A composition comprising amylase and an inert carrier safe for ruminant animal consumption, wherein said amylase is from Aspergillus oryzae.

12. The composition of claim 11 further comprising grain or total mix ration.

13. The composition of claim 12, wherein the amylase is at a specific activity of 600 units per gram of the composition.
